(19) United States
(12) Patent Application Publication
(10) Pub. No.: US 2021/0340156 A1
(43) Pub. Date: Nov. 4, 2021

(54) PROCESS FOR PREPARATION OF ERIBULIN AND INTERMEDIATES THEREOF

(71) Applicant: DR. REDDY'S LABORATORIES LIMITED, Hyderabad (IN)

(72) Inventors: Graham Andrew MEEK, Cambridgeshire (GB); Philip Mark JACKSON, Cambridgeshire (GB); Thomas MAHONEY, Cambridgeshire (GB); Pieter David DE KONING, Saffron Walden, Essex (GB); Robert Wen Ming DAVIDSON, Cambridgeshire (GB); Christopher James COBLEY, Cambridgeshire (GB); David Andrew CHAPLIN, Cambridgeshire (GB); Helen SAMUEL, Edmunds Suffolk (GB); Achanta SRINIVAS, Hyderabad (IN); Kurella SRINIVAS, Hyderabad (IN); Madaraboina MAHENDER, Hyderabad (IN)

(21) Appl. No.: 17/283,421

(22) PCT Filed: Oct. 4, 2019
(86) PCT No.: PCT/IB2019/058456

§ 371 Date: Apr. 7, 2021

(30) Foreign Application Priority Data
Oct. 9, 2018 (IN) 201841038308
Nov. 28, 2018 (IN) 201841044912

(51) Int. Cl. C07D 493/22

(52) U.S. Cl. CPC C07D 493/22; C07B 2200/13

(57) ABSTRACT

The present application relate to crystalline azide compound of formula (II) which is used as an intermediate for the preparation of halichondrin B analogues such as eribulin or pharmaceutically acceptable salts thereof. The present application also covers purification process of azide compound of formula (II) and its further conversion to eribulin or a pharmaceutically acceptable salts thereof.

### INTRODUCTION

Aspects of the present application relate to crystalline azide compound of formula (II) which is used as an intermediate for the preparation of halichondrin B analogues such as eribulin or pharmaceutically acceptable salts thereof and its purification process.

The drug compound having the adopted name eribulin, is a synthetic analogue of halichondrin B, and is represented by structure of formula I.

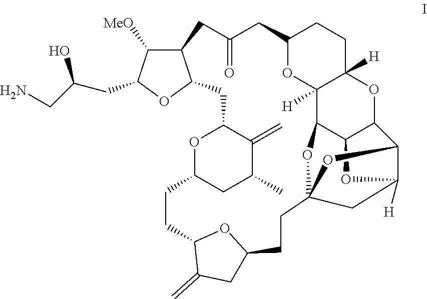

Eribulin is a microtubule inhibitor indicated for the treatment of patients with metastatic breast cancer who have previously received at least two chemotherapeutic regimens for the treatment of metastatic disease. U.S. Pat. No. 6,214,865 discloses eribulin and its pharmaceutically acceptable salts.

Processes for the preparation of Eribulin are described in U.S. Pat. No. 6,214,865, PCT publication Nos. WO 2005/118565A1, WO 2009/124237A1, WO 2015/000070A1 and WO 2015/085193A1.

U.S. Pat. No. 6,214,865 discloses azide compound of formula II which is used as the penultimate intermediate for the preparation of halichondrin B analogues such as eribulin and its pharmaceutically acceptable salt.

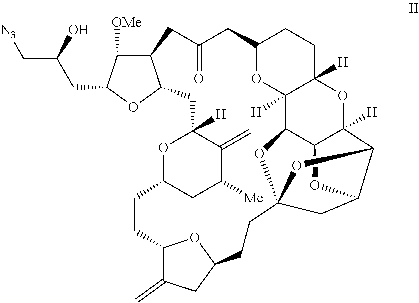

Some of the impurities are known to be unusually potent or to produce toxic or unexpected pharmacological effects. The US Food and Drug Administration (FDA) as well as European Medicines Agency guidance suggest that the API is free from impurities to the maximum possible extent. The present application provides azide compound of formula (II) in crystalline form and process for its preparation. Isolation of crystalline intermediates provide certain advantages like reducing the need of multiple purifications, increasing the conversion rate and reducing the formation of carryforward by products or impurities in the subsequent step which in turn lead to increase the overall yield in the synthesis.

### SUMMARY

In the first embodiment, the present application provides a crystalline compound of formula (II)

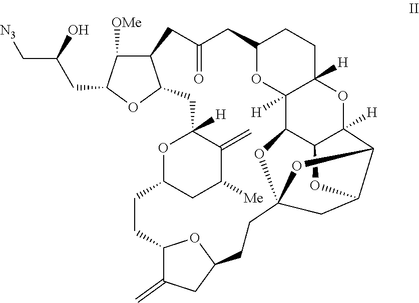

In the second embodiment, the present application provides a crystalline compound of formula (II) characterized by its powder X-ray diffraction (PXRD) pattern having peaks at about 9.9±0.2, 10.8±0.2, 13.1±0.2, 13.8±0.2, 14.5±0.2, 15.0±0.2, 16.4±0.2, 17.3±0.2, 19.0±0.2, 20.8±0.2, 21.2±0.2 and 22.2±0.2 degrees 2theta.

In the third embodiment, the present application provides a compound of formula (II) characterized by its PXRD pattern as illustrated by FIG. 1.

In the fourth embodiment, the present application provides a process for the preparation of crystalline compound of formula (II), said process comprising:

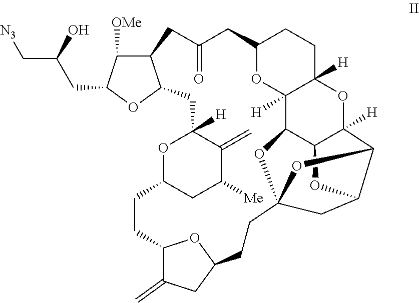

(a) dissolving compound of formula (II) in a solvent or mixture of solvents,
(b) optionally adding seed crystals, and
(c) isolating crystalline compound of formula (II)

In the fifth embodiment, the present application provides purification method for compound of formula (II),

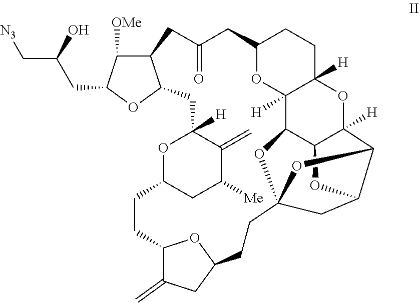

said method comprising purifying of crude compound of formula (II) using one or more methods selected from isolation, slurrying in a suitable solvent, liquid-liquid extraction, chromatography and treating with adsorbent.

In the sixth embodiment, the present application provides substantially pure compound of formula (II) having a purity of at least 96.0% by HPLC or UPLC obtained by a process of the present application.

In the seventh embodiment, the present application provides process for preparation of eribulin or a pharmaceutically acceptable salt thereof, said process comprising:
(a) converting crystalline compound of formula (II) to eribulin; and

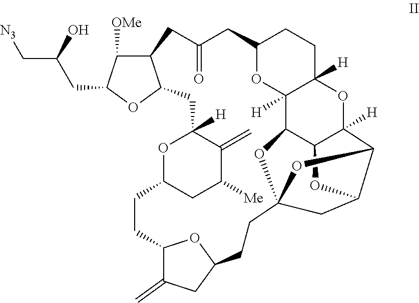

(b) optionally converting eribulin to pharmaceutically acceptable salt of eribulin.

### BRIEF DESCRIPTION OF THE DRAWING

FIG. 1 is an illustration of powder X-ray diffraction (“PXRD”) pattern of compound of formula (II) prepared according to example 7

FIG. 2 is an illustration of the asymmetric unit of compound of formula (II) crystal prepared according to example 3.

### DETAILED DESCRIPTION

In an aspect, the present application provides a process for preparation of crystalline compound of formula (II), Suitable solvents that may be used for dissolving compound of formula (II) and isolating crystalline compound of formula (II) include one or more solvents selected from water, alcohols, ketones, hydrocarbons, halogenated hydrocarbons, esters, ethers, polar aprotic solvents, nitriles or any mixtures thereof.

In another aspect, the present application provides purification of compound of formula (II). Purification of compound of formula (II) may be carried out by one or more methods selected from isolation, slurrying in a suitable solvent, liquid-liquid extraction, chromatography and treating with adsorbents.

Suitable isolation methods that may be used for purification of compound of formula (II) include decantation or filtration or precipitation from a solvent or precipitation by adding an anti-solvent to a solution or by evaporation of solution and the like or any other suitable isolation techniques known in the art. Optionally the said precipitation may result in a crystalline compound including solvates and hydrates thereof. Suitable solvents that may be used for said isolation include water, alcohols, ketones, hydrocarbons, halogenated hydrocarbons, esters, ethers, polar aprotic solvents, nitriles or any mixtures thereof.

Suitable solvents that may be used for purification of compound of formula (II) by slurrying in a suitable solvent include water, alcohols, ketones, hydrocarbons, halogenated hydrocarbons, esters, ethers, polar aprotic solvents, nitriles or any mixtures thereof.

Purification of compound of formula (II) may be carried out by liquid-liquid extraction. In the said process, the compound is dissolved in a suitable first solvent to obtain a solution and the resulting solution is washed with a second solvent that is immiscible with the solution and the pure compound is isolated from the solution obtained after said washing.

Suitable chromatographic techniques that may be used for purification of compound of formula (II) are selected from column chromatography, flash chromatography, ion exchange chromatography, supercritical fluid chromatography, high performance liquid chromatography (both reverse phase and normal phase), expanded bed adsorption chromatography and simulated moving bed chromatography or any combination thereof.

Suitable solvents that may be used in the chromatographic techniques include water, alcohols, ketones, hydrocarbons, halogenated hydrocarbons, esters, ethers, polar aprotic solvents, nitriles or any mixtures thereof.

Suitable mobile phases including buffers such as trifluoroacetate, sulfate, phosphate, chloroacetate, formate, acetate, ammonium formate, ammonium bicarbonate, borate, potassium hydrogen phosphate and the like or supercritical gases such as carbon dioxide (CO₂), xenon (Xe), nitrous oxide (N₂O), sulfur hexafluoride (SF₆), ammonia (NH₃), water (H₂O), ethane (C₂H₆), propane (C₃H₈), n-butane (C₄H₁₀) and the like in combination with suitable solvents as outlined above may be used in chromatography techniques for separation of impurities from the crude compounds which in turn give rise to pure compounds.

The chromatographic methods (for example HPLC, UPLC, SFC and the like) that may be followed to measure the purity of eribulin or a pharmaceutically acceptable salt thereof or purification of eribulin or a pharmaceutically acceptable salt thereof involve the use of columns selected from Torus, Restek Biphenyl, YMC Pro C18, Princeton Diol, Acquity CSH Phenyl Hexyl, ZORBAX Rx-SIL, or any other suitable chromatography columns.

Suitable mobile phases and suitable gradient programs may be used depending on the specific impurities that need to be separated.

Suitable resins that may be used as adsorbents in the chromatographic techniques include cation exchange resins, anion exchange resins, chelated resins, synthetic adsorbents, non-ionic resins or combinations thereof. The resins may be lipophilic, hydrophilic and/or hydrophobic in nature.

Purification of compound of formula (II) may be carried out by treating with adsorbents in a batch mode. Suitable adsorbents that may be used for purification of compounds provided in the first and second embodiments include silica gel, activated alumina, molecular sieves, magnesium silicate, synthetic resin, and the like; or any other suitable adsorbents known in the art.

The purification process may be carried out one or more times using one or more purification methods described in the present application to completely remove the impurities or to get the desired purity of eribulin or any pharmaceutically acceptable salt thereof.

### Definitions

The following definitions are used in connection with the present application unless the context indicates otherwise. In general, the number of carbon atoms present in a given group or compound is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions or the like.

An “alcohol” is an organic compound containing a carbon bound to a hydroxyl group. “C₁-C₆ alcohols” include methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, cyclohexanol, phenol, glycerol and the like.

A “hydrocarbon solvent” is a liquid hydrocarbon compound, which may be linear, branched, or cyclic and may be saturated or have as many as two double bonds or aromatic. Examples of “C₅-C₁₅ aliphatic or aromatic hydrocarbons” include n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, petroleum ethers, benzene toluene, ethylbenzene, m-xylene, o-xylene, p-xylene, indane, naphthalene, tetralin, trimethylbenzene, chlorobenzene, fluorobenzene, trifluorotoluene, anisole, C₆-C₁₂ aromatic hydrocarbons and the like.

An “ether” is an organic compound containing an oxygen atom —O— bonded to two other carbon atoms. “C₂-C₆ ethers” include diethyl ether, diisopropyl ether, methyl t-butyl ether, glyme, diglyme, tetrahydrofuran, 2-methyltetrahydrofuran, 1, 4-dioxane, dibutyl ether, dimethylfuran, 2-methoxyethanol, 2-ethoxyethanol, anisole and the like.

A “halogenated hydrocarbon” is an organic compound containing a carbon bound to a halogen. Halogenated hydrocarbons include dichloromethane, 1,2-dichloroethane, trichloroethylene, perchloroethylene, 1,1,1-trichloroethane, 1,1,2-trichloroethane, chloroform, carbon tetrachloride and the like.

An “ester” is an organic compound containing a carboxyl group —(C═O)—O— bonded to two other carbon atoms. “C₃-C₁₀ esters” include ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate and the like.

A “ketone” is an organic compound containing a carbonyl group —(C═O)— bonded to two other carbon atoms. “C₃-C10 ketones” include acetone, ethyl methyl ketone, diethyl ketone, methyl isobutyl ketone, ketones and the like.

A “nitrile” is an organic compound containing a cyano —(C≡N) bonded to another carbon atom. “C₂-C₆ Nitriles” include acetonitrile, propionitrile, butanenitrile and the like.

A “polar aprotic solvents” include N, N-dimethylformamide, N, N-dimethylacetamide, dimethylsulfoxide, sulfolane, N-methylpyrrolidone and the like;

“Substantially pure” as used herein refers to a compound having a purity of not less than 96.0% or less than 97.5% or not less than 98.0% or not less than 98.5% or not less than 99.0% or not less than 99.5% or not less than 99.7% or not less than 99.8% or not less than 99.9% as measured by a suitable HPLC or UPLC method.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present application. While particular aspects of the present application have been illustrated and described, it would be obvious to those skilled in the art that various other changes and modifications can be made without departing from the spirit and scope of the invention. It is therefore intended to cover in the appended claims all such changes and modifications that are within the scope of this application.

### EXAMPLES

### Example-1: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

A solution of (1S,3S,6S,9S,12S,14R,16R,18S,20R,21R,22S,26R,29S,31R,32S,33R,35R,36S)-20-[(25)-2-hydroxy-3-tosylpropyl]-21-methoxy-14-methyl-8,15-bis(methylene)-2,19, 30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (737 mg) in anhydrous N,N-dimethylformamide (11 mL) was added to a solution of tetra-n-butylammonium azide (890 mg) in anhydrous N,N-dimethylformamide (4.6 mL) under a nitrogen atmosphere. The resultant reaction mass was heated to 65° C. and stirred at this temperature for 4 hours. The solution was then cooled to ambient temperature. Water (15 mL) and tert-butyl methyl ether (MTBE, 15 mL) were sequentially added and the solution was transferred to a separating funnel, washing across with further MTBE (2×5 mL). The phases were separated and the organic phase was further washed with water (3×15 mL). The combined aqueous washes were back extracted with MTBE (10 mL). The combined organic extracts were washed with 10 wt % aqueous sodium chloride solution (10 mL), dried (MgSO₄), filtered and the filtrate concentrated in vacuo. The residue was purified by flash column chromatography: (Combiflash; eluent: heptane/ethyl acetate gradient elution). Fractions containing the product were combined and concentrated in vacuo. The resultant compound was dissolved in acetonitrile. The concentrated solution of title compound in acetonitrile was purified using supercritical fluid chromatography on a Princeton Diol column using carbon dioxide and acetonitrile as a mobile phase. Fractions containing the product were combined and concentrated to give title compound (Purity by UPLC: 98.55%).

### Example-2: Crystallization of (1S,3S,6S,9S,12S,14R,16R,18S,20R,21R,22S,26R,29S, 31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one

MTBE (1.6 mL) and heptane (1 mL) were sequentially added to crude (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37, 39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (133 mg) to provide a clear solution. A seed crystal was added to the solution and the mixture was stirred at 21° C. for 18 hours. The resulting precipitated solid was isolated by filtration and then washed with heptane (2×1 mL). The solid was then dried in vacuo under a flow of nitrogen to provide a white crystalline solid (Purity by UPLC: 97.8%).

### Example-3: Preparation of (1S,3S,6S,9S,12S,14R,16R,18S,20R,21R,22S,26R,29S, 31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one single crystals

(1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19, 30,34,37,39,40,41-octaoxanonacyclo[24.9.2.1^{3,32}.1^{3,33}.1^{6,9}.1^{12,16}.0^{18,22}.0^{29,36}.0^{31,35}]hentetracontan-24-one (16 mg) was dissolved in MTBE (0.3 mL) and pentane (0.2 mL) was subsequently added. The solvents were allowed to slowly evaporate to afford crystals.

### Single Crystal X-Ray Determination:

A small portion of this sample was suspended in perfluoroether oil; a colourless cut block-shaped crystal of size 0.300×0.138×0.100 mm³ was selected and mounted on a MITIGEN holder with perfluoroether oil then aligned upon a Rigaku 007HF diffractometer, equipped with Varimax confocal mirrors and an AFC11 goniometer and HyPix 6000 detector. The crystal was kept at a steady T=100(2) K during data collection. The structure was solved with the ShelXT (Sheldrick, 2015) structure solution program using the Intrinsic Phasing solution method and by using Olex2 (Dolomanov et al., 2009) as the graphical interface. The model was refined with version 2018/3 of ShelXL (Sheldrick, 2015) using Least Squares minimisation.

### Crystal Data:

Molecular Formula C₄₀H₅₇N₃O₁₁
Molecular weight 755.88
Crystal System Monoclinic
Space Group C2 (No. 5)
Unit cell dimensions: a=17.54780(10) Å
   b=9.39920(10) Å
   c=24.1651(2) Å
   β=104.6430(10)°
   α=γ=90′
Wavelength/Å 1.54178
Volume 3856.22(6) Å³

### Example-4: Purification of (1S,3S,6S,9S,12S,14R,16R,18S,20R,21R,22S,26R,29S, 31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one

Crude compound of formula (II) (768 mg) was dissolved in MTBE (7.7 mL) and heptane (5.0 mL) was added. Seed crystal of compound of formula (II) was added and stirred for 24 hours. Separated solid was filtered, washed with heptane (2×5.0 mL) to yield purified compound of formula (II).

### Example-5: Purification of (1S,3S,6S,9S,12S,14R,16R,18S,20R,21R,22S,26R,29S, 31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one

Crude compound of formula (II) (33 mg) was dissolved in acetone (330 μL) to give a clear, colourless solution. Water was added in 100 μL aliquots until a persistent haze was seen (300 μL was added). A seed crystal was added, the flask was sealed and the mixture was held at ambient temperature overnight. The crystalline solid was isolated by filtration to give purified title compound (Purity by UPLC: 97.7%).

### Example-6: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

Charged (1S,3S,6S,9S,12S,14R,16R,18S,20R,21R,22S,26R,29S,31R,32S,33R,35R,36S)-20-[(25)-2-hydroxy-3-tosylpropyl]-21-methoxy-14-methyl-8,15-bis(methylene)-2,19, 30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (2.5 g), toluene (37.5 mL) and tetra-n-butylammonium azide (2.74 g) into a round bottom flask under nitrogen atmosphere. The resultant reaction mass was heated to 65° C. and stirred at this temperature for 29 hours. Water (37.5 mL) was added at ambient temperature and stirred for 15 minutes. The phases were separated and aqueous phase was extracted with toluene (2×20 mL). Combined organic phase was dried over MgSO₄ and concentrated in vacuo. The crude compound was purified by flash column chromatography: (Combiflash; eluent: heptane/ethyl acetate gradient elution). Fractions containing the product were combined and concentrated to give title compound (Purity by HPLC: 98.07%).

### Example-7: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

Charged (1S,3S,6S,9S,12S,14R,16R,18S,20R,21R,22S,26R,29S,31R,32S,33R,35R,36S)-20-[(2S)-2-hydroxy-3-tosylpropyl]-21-methoxy-14-methyl-8,15-bis(methylene)-2,19, 30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (1.85 g), toluene (31.5 mL) and tetra-n-butylammonium azide (1.901 g) into a round bottom flask under nitrogen atmosphere. The resultant reaction mass was heated to 65° C. and stirred at this temperature for 35 hours. Water (18.5 mL) was added at ambient temperature and stirred for 15 minutes. The phases were separated and aqueous phase was extracted with toluene (2×9.25 mL). Combined organic phase was dried over MgSO₄ and concentrated in vacuo. The crude compound was purified by flash column chromatography: (Combiflash; eluent: heptane/ethyl acetate gradient elution). Fractions containing the product were combined, concentrated and recrystallized from MTBE and heptane to give title compound (Purity by HPLC: 99.63%).

### Example-8: Preparation of Eribulin

Triphenylphosphine (97 mg) was added under nitrogen to a solution of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37, 39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (253 mg) in THF (2.5 mL) and water (0.25 mL). After stirring the solution at 21° C. for 24 hours, dichloromethane (10 mL) and 9:9:182 w:w:w sodium bicarbonate:sodium carbonate:water solution (5 mL) were added and the phases separated. The aqueous phase was extracted with dichloromethane (2×5 mL). The combined dichloromethane phases were dried (K₂CO₃), filtered and concentrated in vacuo at a temperature <35° C. The residue was purified by flash column chromatography (Combiflash; Eluent: dichloromethane/methanol/ammonium hydroxide). The fractions containing product were combined and concentrated in vacuo. The resulting residue was dissolved in anhydrous dichloromethane/pentane (3:1 v/v, 4.2 mL), filtered under vacuum with a nitrogen purge over the funnel. The residue on the filter was then washed with further anhydrous dichloromethane/pentane (3:1 v/v, 2×1 mL). The resultant solution was concentrated in vacuo to provide the title compound as a white solid (purity by HPLC: 99.4%).

### Example-9: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

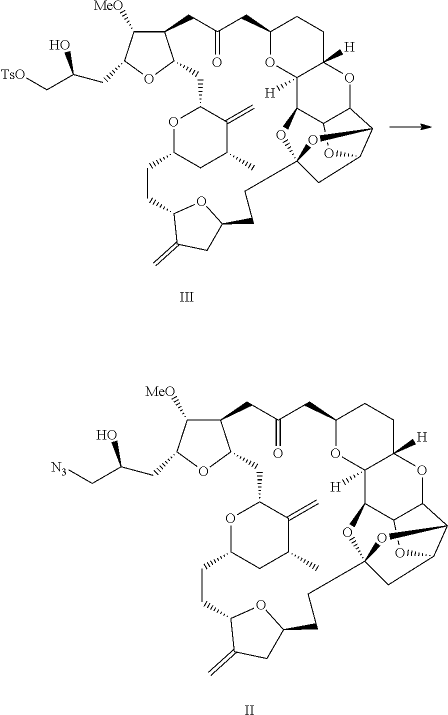

A solution of (1S,3S,6S,9S,12S,14R,16R,18S,20R,21R,22S,26R,29S,31R,32S,33R,35R,36S)-20-[(2S)-2-hydroxy-3-tosylpropyl]-21-methoxy-14-methyl-8,15-bis(methylene)-2,19, 30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (200 mg) in MTBE (1.0 mL) was added to a suspension of sodium azide (61 mg), triethylbenzylammonium chloride (27 mg) and toluene (4.0 mL). The reaction mixture was heated to 60° C. and stirred at 60° C. for 20 hours. Water (10 mL) was added at 21° C. and the biphasic mixture was separated. The organic layer was washed with water (10 mL) and the combined aqueous layers extracted with toluene (10 mL) and MTBE (5 mL). The combined organic layer washed with water (10 mL), brine (10 mL), dried with MgSO4 and concentrated in vacuo. The resulting oil was dissolved in DCM (5 mL) and heptane (10 mL) then concentrated in vacuo to yield a white solid. The crude solid was purified by flash column chromatography (0-100% MTBE in DCM), the product-containing fractions combined and concentrated in vacuo as above including DCM/heptane treatment to yield a white solid. The solid was dissolved in MTBE (2.0 mL) and heptane (1.25 mL) added to provide a clear solution that was seeded with a previously obtained crystal of title compound. The solution was stirred for 20 hours, separated solid was isolated by filtration to yield title compound as a white crystalline solid.

### Example-10: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

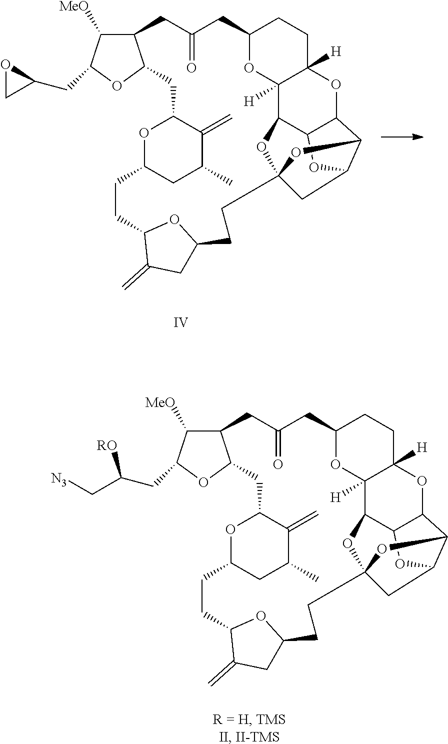

A solution of epoxy compound of formula IV (0.3 M in MTBE, 0.4 mL) was added to (R,R)-N,N′-bis(3,5-di-tert-butylsalicylidene)-1,2-cyclohexanediaminochromium(III) chloride (3.8 mg) followed by TMSN₃ (32 μL). The reaction mixture was stirred at 21° C. for 24 hours. The reaction was quenched with NH₄Cl (sat. aq.) (0.5 mL) then partitioned between MTBE (5 mL) and 1 M HCl (aq.) (5 mL). The biphasic mixture was separated. The aqueous layer was extracted with MTBE (5 mL) and the combined organic layer was washed with water (5 mL), NaHCO₃(sat. aq.) (5 mL), brine (5 mL), dried (MgSO₄), filtered and concentrated in vacuo to afford the title compound. The resulting compound was purified by flash column chromatography (0-100% MTBE in DCM) to separate compound of formula II and formula II-TMS.

### Example-11: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

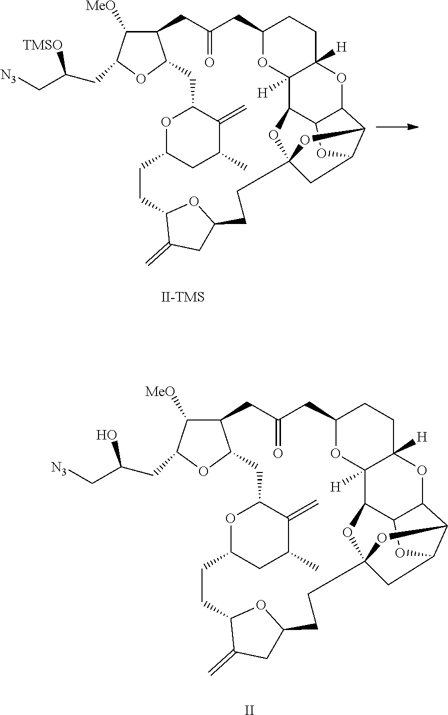

Compound of formula II-TMS (100 mg) was dissolved in THF (1.0 mL). Acetic acid (8 mg) and 1.0 M TBAF in THF (0.13 mL) were added and the reaction mixture and stirred at 21° C. for 18 hours. The reaction mixture was partitioned between MTBE (5 mL) and water (5 mL). The biphasic mixture was separated. The aqueous layer was extracted with MTBE (5 mL) and the combined organics washed with water (5 mL), brine (5 mL), dried (MgSO₄) and filtered. The filtrate was concentrated in vacuo. The resulting residue was purified by flash column chromatography (0-100% MTBE in DCM), the product-containing fractions combined and concentrated in vacuo to yield a colourless oil which was dissolved in DCM (5 mL) and heptane (10 mL). The resulting solution was concentrated in vacuo to yield title compound as a white solid.

### Example-12: Preparation of (1S,3S,6S,9S,12S,14R,16R,18S,20R,21R,22S,26R,29S,31R,32S,33R,35R,36S)-20-[(2S)-3-azido-2-hydroxypropyl]-21-methoxy-14-methyl-8,15-bis(methylene)-2,19,30,34,37,39,40,41-octaoxanonacyclo [24.9.2.1^{3,32}.1^{3,33}.1^{6,9}.1^{12,16}.0^{18,22}.0^{29,36}.0^{31,35}]hentetracontan-24-one (Formula II)

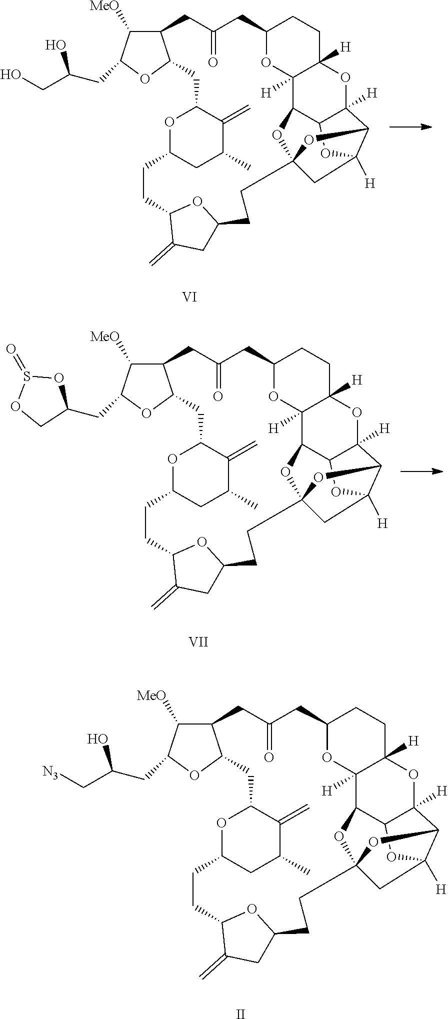

Triethylamine (122 μL) was added to the solution of compound of formula VI (160 mg) in dichloromethane (3.1 mL) and the resultant reaction mass was cooled to −10 and −20° C. Thionyl chloride (0.31 mL of a 1M solution in dichloromethane) was slowly added at −10 and −20° C. and stirred for 1 hour. MTBE (5 mL) and saturated ammonium chloride solution (4 mL) was added and the mixture warmed to room temperature. Phases were separated, aqueous phase was extracted with MTBE (5 mL). The combined organic extracts were dried over MgSO₄ and concentrated in vacuo and the residue purified by flash column chromatography (Combiflash; eluent: heptane/ethyl acetate) to provide the compound of formula VII as an approximately 1:1 mixture of diastereoisomers at sulfur (152 mg) as a tan solid.

To the compound of formula VII (152 mg) THF (2 mL) and Tetrabutylammonium azide (556 mg) was added and the resultant reaction mass was stirred at room temperature for 8 days. Water (5 mL) and MTBE (5 mL) was added to the reaction mass and stirred for 15 minutes. Phases were separated and aqueous phase was extracted with MTBE (5 mL). The combined organic extracts were dried over MgSO₄ and concentrated in vacuo. The resultant residue purified by flash column chromatography (Combiflash; eluent: dichloromethane/MTBE) to provide the title compounds as a 94.4:5.6 mixture of regioisomers (123 mg) as a white solid.

### Example-13: Preparation of Eribulin

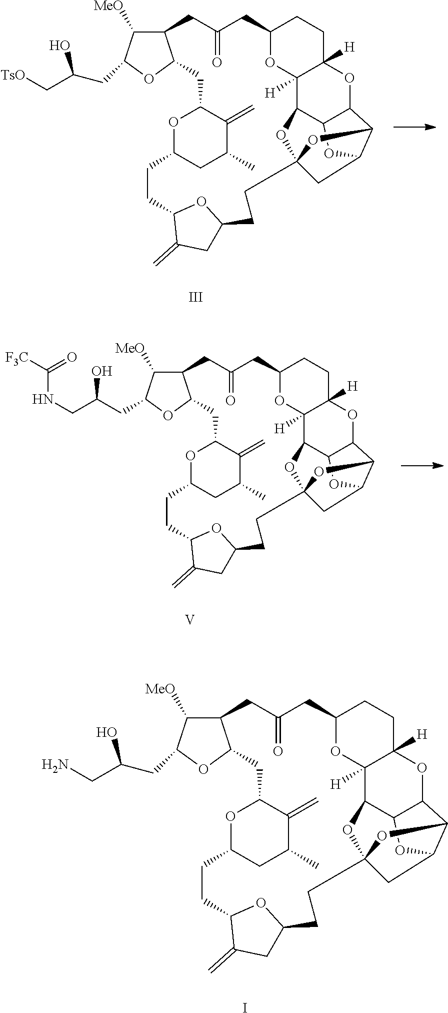

Compound of formula (III) (100 mg) was dissolved in DMF (0.5 mL) and added to trifluoroacetamide (77 mg) followed by 1.0 M potassium tert-butoxide in THF (0.7 mL). The reaction mixture was heated to 60° C. and stirred at 60° C. for 40 hours. The reaction mixture was cooled to 21° C. then partitioned between MTBE (5 mL) and NH₄Cl (sat. aq.) (5 mL). The biphasic mixture was separated. The aqueous layer was extracted with MTBE (2×5 mL) and the combined organic layers were washed with water (5 mL), brine (5 mL), dried (MgSO₄), filtered and concentrated in vacuo. The resulting residue was purified by flash column chromatography (0-100% MTBE in DCM), the product-containing fractions combined and concentrated in vacuo to yield compound of formula V as a white solid.

The resultant white solid (52 mg) was dissolved in DCM (0.5 mL) and MeOH (0.5 mL). To this solution was added K₂CO₃ (21 mg) and the reaction mixture stirred at 21° C. for 48 hours. The reaction mixture was diluted with MTBE (5 mL) filtered and concentrated in vacuo. The resulting residue was purified by flash column chromatography (DCM: MeOH:NH₄OH (aq)), the product-containing fractions combined and concentrated in vacuo to yield title compound as a white solid.

1. Crystalline compound of formula (II)

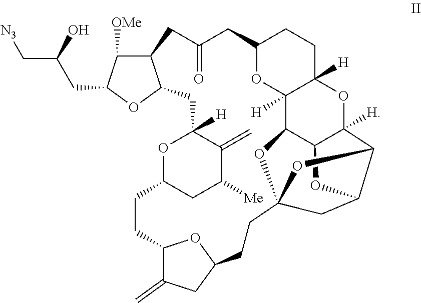

2. The crystalline compound of claim 1, wherein the crystalline compound is characterized by powder X-ray diffraction (PXRD) pattern having peaks at about 9.9±0.2, 10.8±0.2, 13.1±0.2, 13.8±0.2, 14.5±0.2, 15.0±0.2, 16.4±0.2, 17.3±0.2, 19.0±0.2, 20.8±0.2, 21.2±0.2 and 22.2±0.2 degrees 2theta.

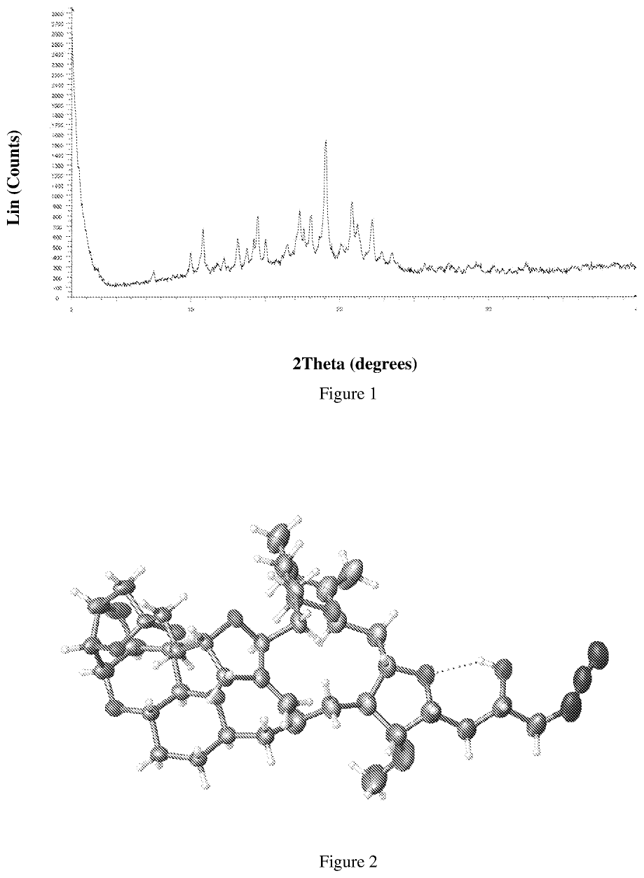

3. The crystalline compound of claim 1, wherein the crystalline form has powder X-Ray diffraction (PXRD) pattern substantially the same as PXRD pattern as illustrated by FIG. 1.

4. A process for the preparation of crystalline compound of claim 1, said process comprising:
(a) dissolving compound of formula (II) in a solvent or mixture of solvents,
(b) optionally adding seed crystals, and
(c) isolating crystalline compound of formula (II).

5. The process according to claim 4, wherein the solvent is selected from water, alcohols, ketones, hydrocarbons, halogenated hydrocarbons, esters, ethers, polar aprotic solvents, nitriles or mixtures thereof.

6. The process according to claim 4, wherein the solvent is selected from methyl tertiary butyl ether, ethyl acetate, n-heptane, acetonitrile, toluene, pentane or mixtures thereof.

7. A process for purification of compound of formula (II), said process 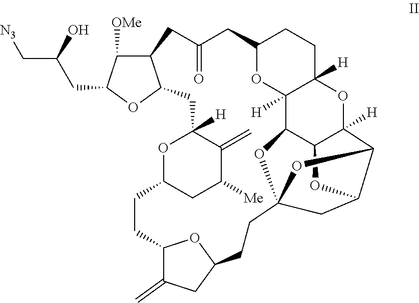
comprising purifying crude compound of formula (II) using one or more methods selected from isolation, slurrying in a suitable solvent, liquid-liquid extraction, chromatography and treating with adsorbent.

8. The process according to claim 7, wherein the crude compound of formula (II) Is purified using chromatographic methods.

9. The process according to claim 8, wherein the crude compound of formula (II) is purified using flash chromatography or ion exchange chromatography or supercritical fluid chromatography or high performance liquid chromatography.

10. The process according to claim 7, wherein the crude compound of formula (II) is purified by isolating from a solvent or by slurrying in a solvent.

11. The process according to claim 7, wherein the crude compound of formula (II) is purified by crystallizing from a solvent or mixture of solvents.

12. Substantially pure compound of formula (II), wherein the purity of compound 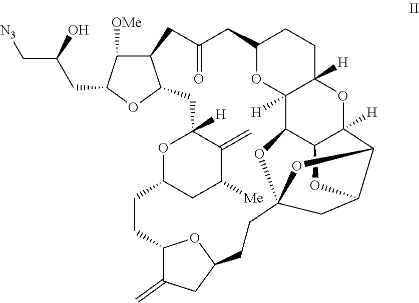
of formula (II) is at least 96% as measured by HPLC or UPLC.

13. The substantially pure compound of claim 12, wherein the purity of formula (II) is at least 97% as measured by HPLC or UPLC.

14. A process for preparation of eribulin or a pharmaceutically acceptable salt thereof from a compound of claim 1, said process comprising:
(a) converting compound of formula (II) to eribulin; and
(b) optionally converting eribulin to Pharmaceutically acceptable salt of eribulin.

15. A process for preparation of eribulin or a pharmaceutically acceptable salt thereof from the compound obtained from process of claim 4, said process comprising:
(a) converting crystalline compound of formula (II) to eribulin; and
(b) optionally converting eribulin to pharmaceutically acceptable salt of eribulin.
